(19) United States
(12) Patent Application Publication
(10) Pub. No.: US 2026/0013932 A1
(43) Pub. Date: Jan. 15, 2026

(54) EXPANDABLE CATHETER WITH A COIL-SPRING ASSEMBLY

(71) Applicant: BIOSENSE WEBSTER (ISRAEL) LTD., Yokneam (IL)

(72) Inventors: CHRISTOPHER THOMAS BEECKLER, BREA, CA (US); JOSEPH THOMAS KEYES, SIERRE MADRE, NJ (US); ISHAN KAMAL KHAN, YORBA LINDA (BD); KEVIN OKARSKI, IRWINDALE, CA (US)

(21) Appl. No.: 18/766,871

(22) Filed: Jul. 9, 2024

(51) Int. Cl. A61B 18/14; A61B 18/00; A61B 34/20

(52) U.S. Cl. CPC A61B 18/1492; A61B 34/20; A61B 2018/00077; A61B 2018/0016; A61B 2018/00172; A61B 2018/00184; A61B 2018/00267; A61B 2018/00351; A61B 2018/00577; A61B 2018/00839; A61B 2018/1467; A61B 2018/1497; A61B 2034/2051

(57) ABSTRACT

A catheter comprising: a shaft configured for insertion into a body of a subject; an expandable assembly mounted at a distal end of the shaft and comprising a proximal end, a distal end, and a longitudinal axis extending between the proximal and distal ends; a coil-spring assembly extending between the proximal end and the distal end of the expandable assembly, along the longitudinal axis; the coil spring assembly comprising at least two nested coil springs which define an inner passageway for an elongate element to be passed therethrough; the coil-spring assembly being configured to deflect along with the expandable assembly while the inner passageway is maintained open.

### TECHNOLOGICAL FIELD

The presently disclosed subject matter generally relates to expandable catheters, and more specifically to an expandable catheter which can be used in pulmonary vein isolation (PVI) for atrial fibrillation treatment.

### BACKGROUND

US 2021/0187241 to Govari et al. discloses “One embodiment includes a catheter apparatus, including an elongated deflectable element including a distal end, a coupler connected to the distal end, a pusher including a distal portion, and configured to be advanced and retracted through the deflectable element, a nose connector connected to the distal portion, and including a distal receptacle having an inner surface and a distal facing opening, and an expandable assembly including flexible polymer circuit strips, each strip including electrodes disposed thereon, the strips being disposed circumferentially around the distal portion of the pusher, with first ends of the strips being connected to the coupler and second ends of the strips including respective hinges entering the distal facing opening and connected to the inner surface of the distal receptacle, the strips being configured to bow radially outward when the pusher is retracted expanding the expandable assembly from a collapsed form to an expanded form.”

U.S. Pat. No. 11,878,095B2 to Beecklet et al. discloses “A medical probe includes a shaft, an expandable membrane and a probe maneuvering assembly (PMA). The shaft is configured for insertion into a cavity of an organ of a patient. The PMA is located inside the expandable membrane and includes a first elastic element, which is fitted along a longitudinal axis of the PMA and is configured to expand the membrane by shortening the PMA, and to collapse the membrane by elongating the PMA. The PMA further includes a second elastic element, which surrounds at least a portion of the first elastic element and is configured to deflect the membrane relative to the longitudinal axis.”

### OVERVIEW

A broad aspect of the disclosure relates to a catheter comprising an expandable assembly with a central coil-spring assembly, the coil-spring assembly defining a passageway for an elongate element such as a guidewire to be passed therethrough. In some examples, the expandable assembly has a compliant, flexible structure, which can be manipulated between a collapsed state (used for example during maneuvering of the catheter to the treatment site) and an expanded state (used for example at the treatment site, such as for obtaining contact between the expandable assembly and the treated tissue). The coil-spring assembly extends between the proximal and distal ends of the expandable assembly, constituting the only direct coupling between them which passes centrally along a longitudinal axis of the assembly. In such arrangement, due to the elasticity and deflection ability of the coil-spring assembly, compliance of the expandable assembly is least or not affected by the coil-spring assembly, for example as compared to using a rigid shaft or tube for providing the passageway. By having a coil-spring assembly (e.g. as compared to a rigid tube), maneuverability of the catheter through narrow, often curvy anatomical orifices can be facilitated.

Further, while the coil-spring assembly is configured to deflect as well as potentially allow some radial compression to be applied thereon (e.g. in the collapsed state of the expandable assembly), the coil-spring assembly is resilient enough so that its inner passageway is maintained normally open, enabling advancement of an elongate element therethrough. Deflection of the expandable assembly (e.g. over a bending guidewire) would also deflect the coil-spring assembly along, yet the passageway defined by the coil-spring assembly will be maintained open due to the elasticity of the coil springs. By that, the coil-spring assembly may be advantageous over, for example, a continuous tube such as a polymer tube which when extended would be more likely to collapse radially.

In accordance with some examples of the disclosure, the coil-spring assembly is formed of a shape memory material, e.g. nitinol. In some examples, the coil-spring assembly is integrally formed with the expandable element, for example, the splines and the coil-spring assembly can all be formed from a single nitinol element.

The coil-spring assembly can be generally constructed of a material which would not or only least affect the compliancy of the expandable assembly, as well as enable a closely packed coil structure to prevent an elongate element such as a guidewire from exiting. For example, in case of a relatively stiff expandable assembly, a stainless-steel coil-spring assembly can be used.

In accordance with some examples of the disclosure, the coil-spring assembly comprises multiple coil springs, for example, two coil springs, which are nested within one another. The two springs can include counter-rotated springs, which, when overlaid in the nested configuration, intervene with each other, such that the windings of one spring are located intermediate the windings of the other spring. Such arrangement may be potentially advantageous in that lateral gaps of the coil-spring assembly, as a whole, are small enough to prevent from an elongate element, especially a thin elongate element such as a guidewire, from unintentionally protruding or exiting through the gaps.

It is noted that while an expandable assembly in the form of a basket is described herein, other expandable elements are also contemplated, for example, a balloon, a membrane, a stent, a cage, a flow diverter or other expandable structure or a structure defining an inner volume within which a coil-spring assembly can extend longitudinally. The expandable assembly may or may not have electrodes disposed thereon.

### BRIEF DESCRIPTION OF THE DRAWINGS

In order to better understand the subject matter that is disclosed herein and to exemplify how it may be carried out in practice, examples will now be described, by way of non-limiting example only, with reference to the accompanying drawings, in which:

FIG. 1 is a diagram of a catheter-based electrophysiology mapping and ablation system, according to examples of the presently disclosed subject matter;

FIG. 2 and FIG. 3 are perspective views of a catheter comprising an expandable assembly with a central coil-spring assembly through which a guidewire is passed; the expandable assembly shown in an expanded state, according to examples of the presently disclosed subject matter;

FIG. 4 is a perspective view of the catheter of FIGS. 2 and 3 shown with the expandable assembly in a collapsed state, according to examples of the presently disclosed subject matter;

FIGS. 5A-C are a perspective view (FIG. 5A) and two cross section views (FIGS. 5B-C) of the coil-spring assembly, according to examples of the presently disclosed subject matter; and

FIGS. 6A-B are perspective views of the expandable assembly and the coil-spring assembly, in an expanded state of the expandable assembly (FIG. 6A) and a collapsed state of the expandable assembly (FIG. 6B), according to examples of the presently disclosed subject matter.

### DETAILED DESCRIPTION

In the following detailed description, numerous specific details are set forth in order to provide a thorough understanding. However, it will be understood by those skilled in the art that the presently disclosed subject matter may be practiced without these specific details. In other instances, well-known methods and features have not been described in detail so as not to obscure the presently disclosed subject matter.

Unless specifically stated otherwise, as apparent from the following discussions, it is appreciated that throughout the specification discussions utilizing terms such as “processing”, “computing”, “comparing”, or the like, refer to the action(s) and/or process(es) of a computer that manipulate and/or transform data into other data, said data represented as physical, such as electronic, quantities and/or said data representing the physical objects. The term “computer” should be expansively construed to cover any kind of hardware-based electronic device with data processing capabilities.

The various illustrative logical blocks, modules, and algorithm steps described in connection with the examples disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing any departure from the scope of the disclosure.

It will also be understood that a system according to the present disclosure may be, at least partly, implemented on a suitably programmed computer. Likewise, the present disclosure contemplates a computer program being readable by a computer for executing the method of the present disclosure. The present disclosure further contemplates a non-transitory computer-readable memory tangibly embodying a program of instructions executable by the computer for executing the method of the present disclosure.

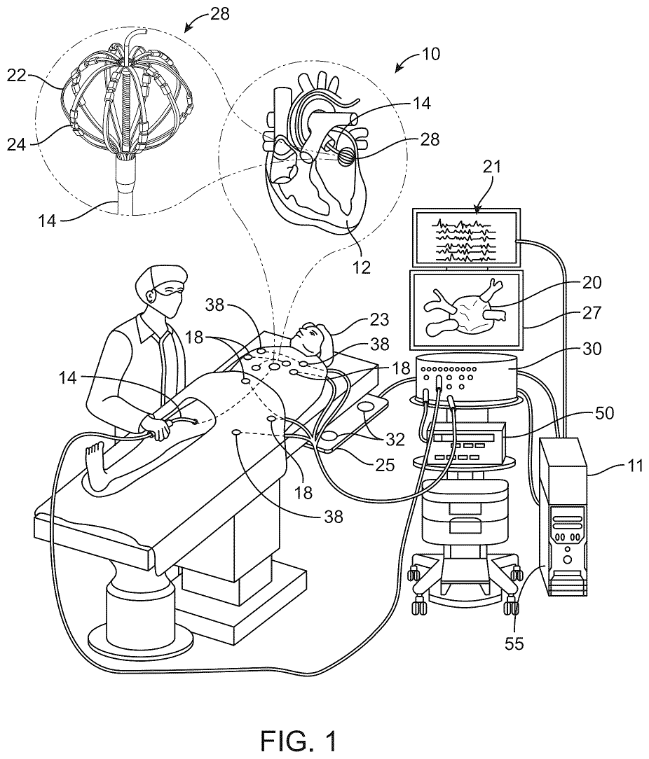

Reference is made to FIG. 1 showing an example catheter-based electrophysiology mapping and ablation system 10. System 10 includes multiple catheters, which are percutaneously inserted by a physician 24 through the patient's vascular system into a chamber or vascular structure of a heart 12. Typically, a delivery sheath catheter is inserted into the left or right atrium near a desired location in heart 12. Thereafter, one or more catheters may be inserted into the delivery sheath catheter so as to arrive at the desired location in heart 12. The plurality of catheters may include catheters dedicated for sensing Intracardiac Electrogram (IEGM) signals, catheters dedicated for ablating and/or catheters dedicated for both sensing and ablating. An example catheter 14 that is configured for ablating tissue and/or for sensing electrical cardiac activity and/or mapping is illustrated herein.

Catheter 14 is an exemplary catheter having an expandable assembly 28 at its distal tip, having one and preferably multiple electrodes 24 optionally distributed over a plurality of flexible spline elements 22. The electrodes 24 are generally configured for delivering ablation energy to tissue, and/or for sensing cardiac electrical signals. The electrodes are generally attached to the splines and secured in place by soldering, welding, or using an adhesive.

Catheter 14 additionally includes one or more position sensors (not shown) embedded in or near the expandable assembly 28 for tracking position and orientation of the assembly. Optionally and preferably, a position sensor is a magnetic based position sensor, for example a position sensor including three magnetic coils for sensing three-dimensional (3D) position and orientation; or a position sensor including one magnetic coil, for sensing a single direction.

Each of the magnetic based position sensors may be operated together with a location pad 25 including a plurality of magnetic coils 32 configured to generate magnetic fields in a predefined working volume. Real time position of distal tip 28 of catheter 14 may be tracked based on magnetic fields generated with location pad 25 and sensed by the magnetic based position sensor. Details of the magnetic based position sensing technology are described in U.S. Pat. Nos. 5,391,199; 5,443,489; 5,558,091; 6,172,499; 6,239,724; 6,332,089; 6,484,118; 6,618,612; 6,690,963; 6,788,967; 6,892,091.

Physician 24 may place the expandable assembly 28 of catheter 14 in contact with the heart wall for sensing a target site in heart 12. For ablation, physician 24 may similarly position the expandable assembly at a target site, and then expand the assembly to bring the electrodes into contact with the tissue intended for ablation. In some examples, the treatment site is selected or identified by sensing, optionally using electrodes of the catheter, cardiac activity patterns, for example to identify arrythmia (e.g. atrial fibrillation) or other disorders.

In some examples (e.g. as part of a “single-shot pulmonary vein isolation” procedure), the catheter is guided to the atrium, for example to the left atrium. In the left atrium, the catheter may be guided to the ostium of the pulmonary vein. Then, the expanded catheter is positioned to produce an ablation lesion, optionally circumferential, at the ostium. In other examples, the left atrial appendage may be treated, or other cardiac sites.

System 10 includes one or more electrode patches 38 positioned for skin contact on patient 23 to establish location reference for location pad 25 as well as impedance-based tracking of electrodes 26. For impedance-based tracking, electrical current is directed to electrodes 26 and sensed at electrode skin patches 38 so that the location of each electrode can be triangulated via the electrode patches 38. Details of the impedance-based location tracking technology are described in U.S. Pat. Nos. 7,536,218; 7,756,576; 7,848,787; 7,869,865; and 8,456,182.

A recorder 11 records and displays electrograms 21 captured with body surface ECG electrodes 18 and intracardiac electrograms (IEGM) captured with electrodes 26 of catheter 14. Recorder 11 may include pacing capability for pacing the heart rhythm and/or may be electrically connected to a standalone pacer.

System 10 may include an ablation energy generator 50 that is adapted to conduct ablative energy to one or more of electrodes at a distal tip of a catheter configured for ablating. Energy produced by ablation energy generator 50 may include, but is not limited to, radiofrequency (RF) energy or pulsed-field ablation (PFA) energy, including monopolar or bipolar high-voltage DC pulses as may be used to effect irreversible electroporation (IRE), or combinations thereof. In an example, the electrodes of the expandable assembly are configured for bipolar energy delivery, with the two polarities being located on the expandable assembly.

Patient interface unit (PIU) 30 is an interface configured to establish electrical communication between catheters, other electrophysiological equipment, power supply and a workstation 55 for controlling operation of system 10. Electrophysiological equipment of system 10 may include for example, multiple catheters, location pad 25, body surface ECG electrodes 18, electrode patches 38, ablation energy generator 50, and recorder 11. Optionally and preferably, PIU 30 additionally includes processing capability for implementing real-time computations of location of the catheters and for performing ECG calculations.

Workstation 55 includes memory, processor unit with memory or storage with appropriate operating software stored therein, and user interface capability. Workstation 55 may provide multiple functions, optionally including (1) modeling the endocardial anatomy in three-dimensions (3D) and rendering the model or anatomical map 20 for display on a display device 27, (2) displaying on display device 27 activation sequences (or other data) compiled from recorded electrograms 21 in representative visual indicia or imagery superimposed on the rendered anatomical map 20, (3) displaying real-time location and orientation of multiple catheters within the heart chamber, and (4) displaying on display device 27 sites of interest such as places where ablation energy has been applied. One commercial product embodying elements of the system 10 is available as the CARTO™ 3 System, available from Biosense Webster, Inc., 31A Technology Drive, Irvine, CA 92618.

In some examples, an irrigation module is provided for delivering irrigation fluid, such as saline solution, to the location of treatment. The irrigation module may comprise a pump and an associated fluid tank.

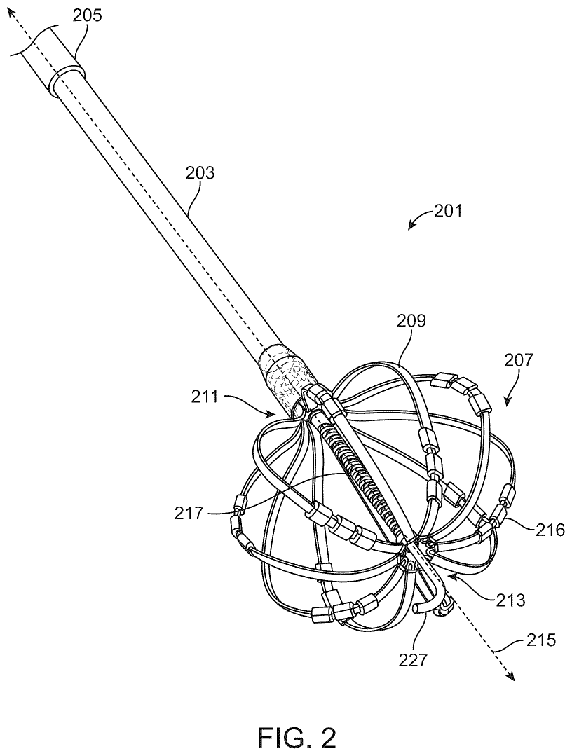

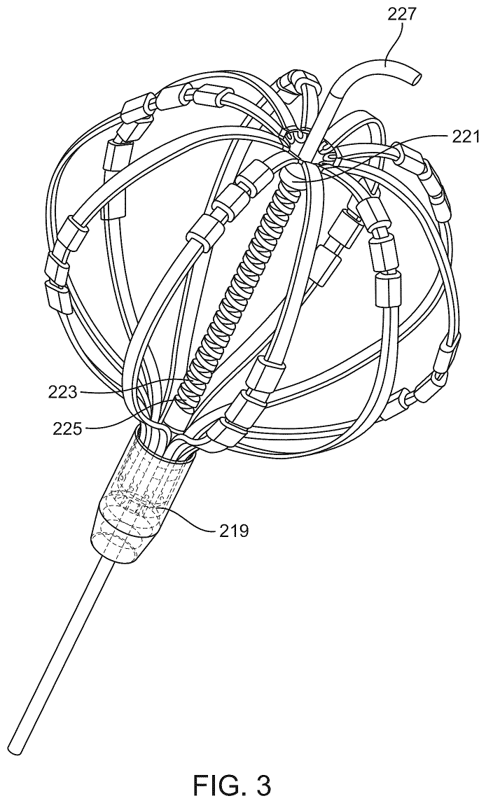

FIG. 2 and FIG. 3 are perspective views of a catheter comprising an expandable assembly with a central coil-spring assembly through which a guidewire is passed; the expandable assembly shown in an expanded state, according to examples of the presently disclosed subject matter.

The exemplary catheter 201 comprises a shaft 203 received within an external delivery sheath 205, via which the catheter can be guided to the treatment site. An expandable assembly 207, for example in the form of a basket, is mounted at a distal end of shaft 203 so as to be extendable from the delivery sheath.

The expandable assembly 207 comprises, in some embodiments, a plurality of flexible splines 209 (e.g. at least 2, 4, 6, 8, 9, 12 or intermediate or larger number of splines). Each spline generally extends between a proximal end 211 and a distal end 213 of the expandable assembly. In the expanded state of the assembly, each spline is configured to bow radially outwardly with respect to a central longitudinal axis 215 of the expandable assembly. The splines may have a thin, substantially flat profile and should be formed of a flexible, resilient material, for example a shape memory alloy such as Nitinol.

The expandable assembly 207 further comprises a plurality of electrodes 216 disposed on at least some of the plurality of splines. The electrodes can be arranged to produce a circumferential ablation lesion in the expanded state of the assembly. As noted, an electrode can be configured for energy delivery, position sensing (e.g. for tissue mapping purposes), or both. In some cases, ablation electrodes and position sensing electrodes are disposed on the expandable assembly and/or on the shaft 203.

As shown, catheter 201 comprises an elongate coil-spring assembly 217 which extends along the longitudinal axis 215, between the proximal end 211 and the distal end 213 of the expandable assembly. At the proximal end, the coil-spring assembly is fixedly received within a proximal coupler 219, to which the splines are also coupled. At the distal end, the coil-spring assembly is fixedly connected to the distal ends of the splines via a distal coupler 221. The proximal and distal couplers are shaped with an opening therethrough, for example formed as cylindrical or funnel shaped couplers. It is noted that a funnel shaped coupler may be advantageous in that it can be smoothly guided into and out from the delivery sheath.

It is noted that in some embodiments the coil-spring assembly can be removably coupled (rather than fixedly coupled) to the distal and/or proximal ends of the expandable assembly. Generally, the coil-spring assembly (at the proximal and/or distal ends thereof) can be welded, glued, or otherwise attached to the respective proximal and distal ends of the splines.

In some embodiments, the coil-spring assembly can be integrally formed with the expandable assembly, for example formed (e.g. cut) from a single nitinol element.

While the splines also extend between the proximal and distal ends of the expandable assembly, the coil spring assembly constitutes the only direct, linear coupling between the distal and proximal ends, extending centrally along longitudinal axis 215. Having no direct linear attachments between the ends of the expandable assembly, other than the coil-spring assembly, may be advantageous since due to the flexibility of the coil-spring assembly, the proximal and distal ends can more freely move with respect to each other, allowing the expandable assembly to maintain its compliance (considering that the splines are thin and flexible).

The coil-spring assembly is generally constructed of helical (coil) springs, such as spring 223 and spring 225. One of the springs is nested within the other, while together defining a normally open inner passageway which extends along the longitudinal axis 215. The defined passageway is large enough in diameter so as to allow an elongate element to be inserted or passed through. In the example shown, a guidewire 227 is inserted through shaft 203 and through coil-spring assembly 217, exiting the expandable assembly via the distal coupler 221. In use, the catheter can be guided over the guidewire to the treatment site. It is noted that elongate elements other than a guidewire are also contemplated, for example a smaller size catheter, a probe (e.g. a sensing probe, an imaging probe), a medication delivery tube, or other elongate element having a diameter smaller than that of the passageway defined by the coil-spring assembly.

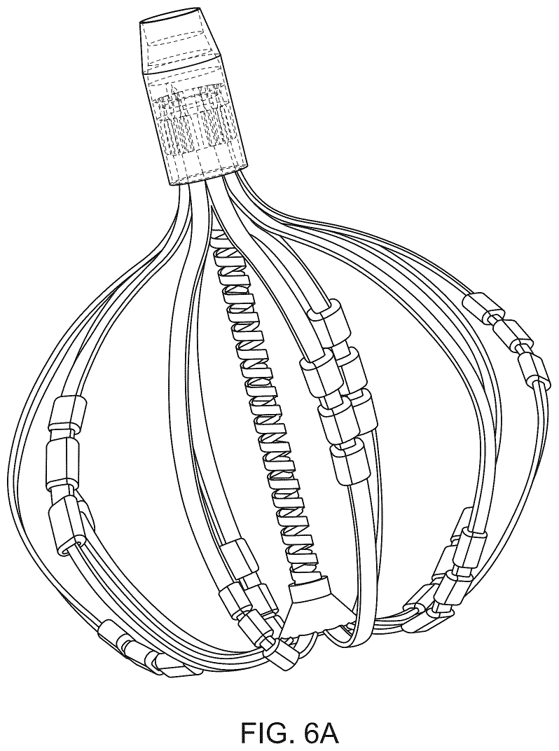

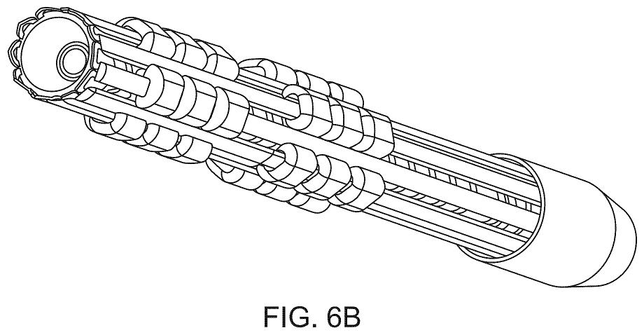

The expandable assembly is further shown in its expanded state and without a guidewire being passed through the coil-spring assembly in FIG. 6A.

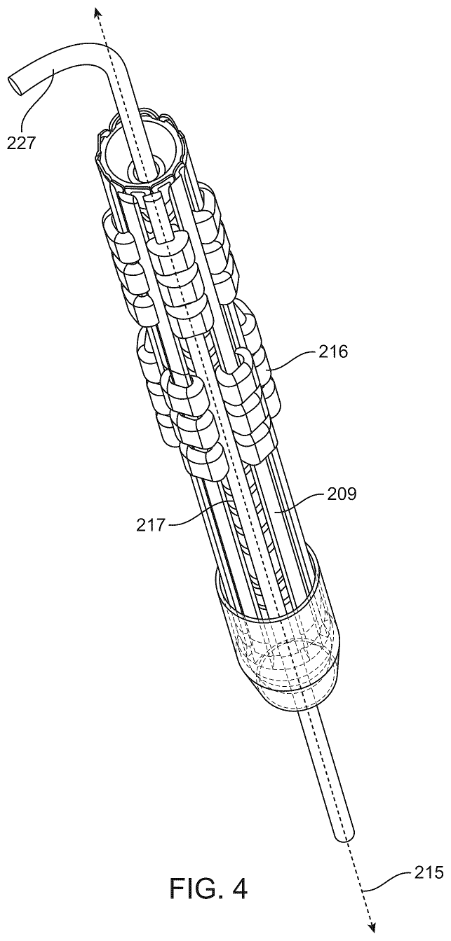

FIG. 4 is a perspective view of the catheter of FIGS. 2 and 3 shown with the expandable assembly in a collapsed state, according to examples of the presently disclosed subject matter. In the collapsed state, the expandable assembly can be introduced via the delivery sheath to the treatment site. Upon being pushed distally from the delivery sheath, the expandable assembly assumes the expanded state.

As shown, each of the collapsed splines 209 assumes a linear configuration, and extends parallel to the longitudinal axis 215. The electrodes 216 of the plurality of splines may be arranged adjacent each other in a circular manner. In the collapsed state of the expandable assembly, and especially during maneuvering of the catheter through narrow and potentially curvy anatomical orifices, the collapsed splines may apply pressure onto the coil-spring assembly 217 (such as in a radially inwards direction); however, the coil-spring assembly is configured be resilient enough and bounce back so that the passageway defined by the coil-spring assembly remains open at least to an extent which allows an elongate member (such as guidewire 227) to pass through.

The expandable assembly is further shown in its collapsed state and without a guidewire being passed through the coil-spring assembly in FIG. 6B.

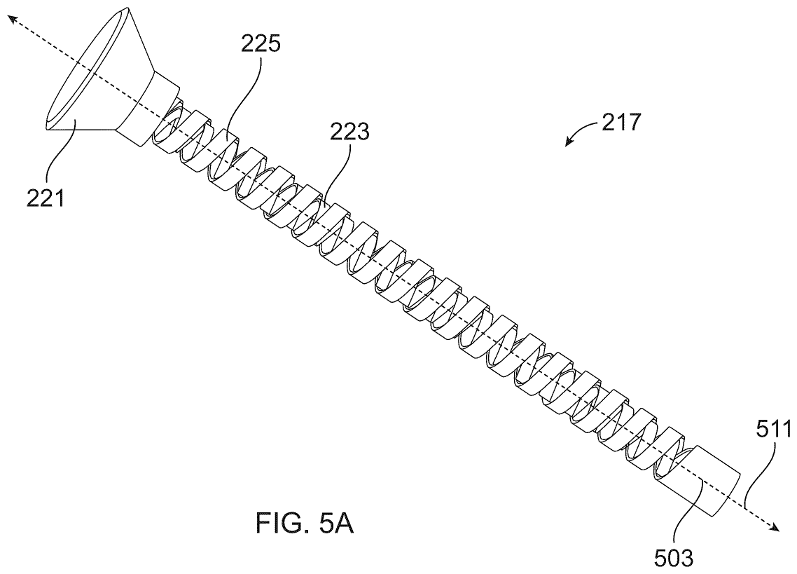

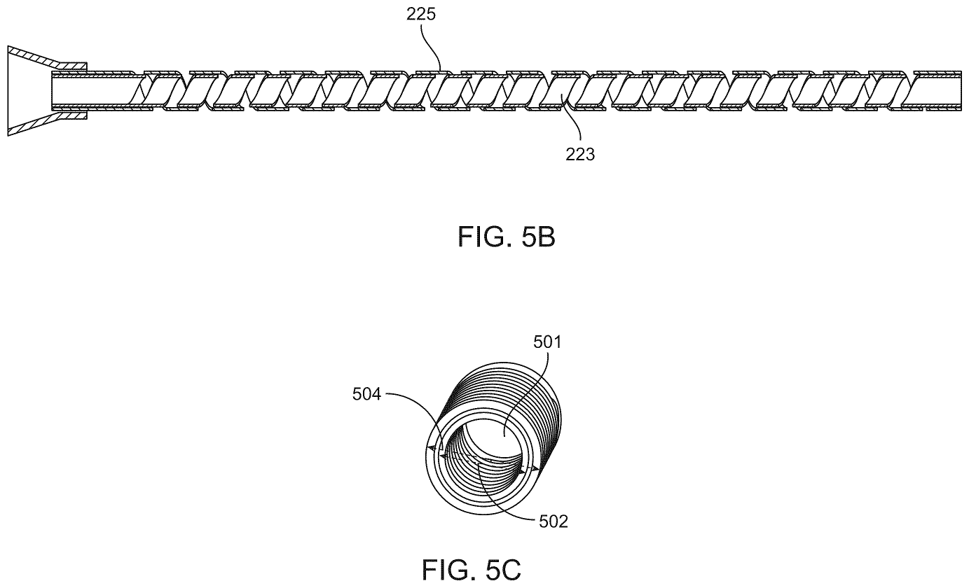

FIGS. 5A-C are a perspective view (FIG. 5A) and two cross section views (FIGS. 5B-C) of the coil-spring assembly, according to examples of the presently disclosed subject matter.

The coil spring assembly 217 is generally comprised of helical springs 223 and 225, with spring 223 being nested within spring 225. An elongate passageway 501 extends between the distal coupler 221 (which connects the coil-spring assembly to the splines, and in this example is funnel shaped) and a proximal end portion 503 of the coil-spring assembly (which is connected to and/or fixedly received within the proximal coupler 219, shown above).

In the example shown, the elongate passageway 501 comprises a substantially circular cross-section profile, shown in FIG. 5C. An inner diameter 502 of the passageway may range between, for example, 0.2 mm-1.3 mm; an outer diameter 504 of the coil spring assembly may range between, for example 0.3 mm-1.6 mm. Non-circular cross section profiles are also to be contemplated.

In some embodiments, as shown in this example, the coil springs 223 and 225 include a left-handed spring (225) and a right-handed spring (223). In such arrangement of nested, counter-rotated springs, windings of the two springs are overlayed and are intervened, such that, for example, a winding of the inner spring 223 passes in between two adjacent windings of the outer spring 225 (when observed in a radial direction). As a result, each of the gaps of the coil spring assembly is effectively smaller than the pitch of each of the springs 223 and 225 separately. This may be advantageous in that a risk of an elongate element such as a guidewire will unintentionally protrude outwardly or exit through one of the gaps (rather than extending via the distal end, as intended) is reduced or prevented. The coil spring assembly can be constructed with small enough lateral gaps so that even in cases in which the elongate element comprises an angled or curved (e.g. J-shaped) tip, the tip will not be able to protrude outwardly from the assembly.

Due to the elastic, flexible nature of the coil springs, the coil spring assembly 217 is configured for at least partial deflection with respect to a longitudinal axis 511 of the coil-spring assembly (which is co-axial with longitudinal axis 215 of the expandable assembly). Although the nested construction of the springs limits the extent of deflection of the assembly as a whole and increases a rigidity of the assembly, for example as compared to each spring separately, the coil-spring assembly can still deflect sufficiently to allow the expandable assembly, especially in its collapsed state, to be at least partially bent or flexed (e.g. with respect to its longitudinal axis). This flexion may be especially advantageous during maneuvering of the catheter through small, narrow anatomical lumens such as veins, cardiac lumens, and the like. Moreover, when guiding a relatively complaint expandable member (such as the described basket) to the target site, the flexible coil-spring assembly does not compromise or only least compromises the compliance of the basket, for example as compared to a rigid shaft or tube (which is commonly used as a lumen for passing a guidewire). Further, the use of counter-rotated springs may contribute to the ability of the coil spring assembly to evenly deflect laterally in different directions (e.g. to have a minimal difference in bending stiffness in the different directions), for example as compared to the use of a single spring.

When considering passageway 501, the coil-spring assembly is sufficiently rigid in a radial direction so that the two springs do not collapse radially inwardly and close the passageway. This may be achieved by a total thickness of a wall of the assembly, measured in a radial direction (the difference between inner diameter 502 and outer diameter 504), being, for example, at least 0.1 mm.

In some cases, one or both of the springs of the coil spring assembly can be formed by cutting a single cylindrical element, e.g. by laser-cutting a nitinol tube.

It is noted that the coil-spring assembly can be constructed of more than two springs, for example, three or four nested springs.

In some cases, the coil spring assembly can comprise of a single spring with a small pitch (in which the coils are densely packed), although this type of structure may be more suitable for use with a large (e.g. thick, high diameter) elongate element which would be at a lower risk of exiting through the lateral gaps.

### SUMMARY

Following is a non-exclusive list of some exemplary examples of the disclosure.

The present disclosure also includes examples which include fewer than all the features in an example and examples using features from multiple examples, even if not listed below.

### EXAMPLE 1

A catheter comprising:
a shaft configured for insertion into a body of a subject;
an expandable assembly mounted at a distal end of the shaft and comprising a proximal end, a distal end, and a longitudinal axis extending between the proximal and distal ends;
a coil-spring assembly extending between the proximal end and the distal end of the expandable assembly, along the longitudinal axis; the coil spring assembly comprising at least two nested coil springs which define an inner passageway for an elongate element to be passed therethrough; the coil-spring assembly being configured to deflect along with the expandable assembly while the inner passageway is maintained open.

### EXAMPLE 2

The catheter according to Example 1, wherein the coil-spring assembly constitutes the only direct coupling between the proximal and distal ends of the expandable assembly which extends centrally along the longitudinal axis.

### EXAMPLE 3

The catheter according to any one of the preceding Examples, wherein the passageway defined by the coil-spring assembly is sized to enable an elongate element in the form of a guidewire to be passed therethrough.

### EXAMPLE 4

The catheter according to Example 1, wherein the coil-spring assembly comprises a lefthanded coiled spring and a righthanded coiled spring.

### EXAMPLE 5

The catheter according to Example 4, wherein a pitch of each of the coil springs is sized so that lateral gaps of the coil-spring assembly are small enough to prevent even a thin elongate element from protruding therethrough.

### EXAMPLE 6

The catheter according to Example 1, wherein deflection of the coil-spring assembly and the expandable assembly is with respect to the longitudinal axis.

### EXAMPLE 7

The catheter according to Example 1, wherein the coil-spring assembly is at least partially compressible and extensible with respect to the longitudinal axis.

### EXAMPLE 8

The catheter according to Example 1, wherein the inner passageway has a circular cross section profile.

### EXAMPLE 9

The catheter according to Example 1, wherein the coil-spring assembly is integrally formed with the expandable assembly.

### EXAMPLE 10

The catheter according to Example 1, wherein the coil-spring assembly is coupled to the proximal and distal ends of the expandable assembly via cylindrical or funnel-shaped members.

### EXAMPLE 11

The catheter according to Example 1, wherein the coil-spring assembly is produced by laser-cutting a nitinol tube.

### EXAMPLE 12

The catheter according to Example 1, wherein the expandable assembly comprises a plurality of electrodes for delivery of ablation energy being mounted thereon.

### EXAMPLE 13

The catheter according to Example 12, wherein the electrodes are configured for pulsed field ablation or RF ablation.

### EXAMPLE 14

The catheter according to Example 1, further comprising a plurality of position sensors disposed on one or both of the shaft and the expandable assembly for tracking a position and an orientation of at least a portion of the catheter.

### EXAMPLE 15

The catheter according to any one of the preceding Examples, wherein the expandable assembly comprises a basket including a plurality of splines, each spline connected to the proximal end and to the distal end; the basket being manipulable between an expanded state in which the splines bow radially outwardly with respect to the longitudinal axis, and a collapsed state in which the splines are aligned parallel to the longitudinal axis.

### EXAMPLE 16

The catheter according to Example 15, wherein in the collapsed state the catheter is sized to be delivered through a delivery sheath; and wherein upon extending the expandable assembly distally from the delivery sheath, the coil-spring assembly extends distally.

### EXAMPLE 17

The catheter according to Example 15, wherein in the collapsed state the plurality of splines extend parallel to the longitudinal axis and thereby parallel to the coil-spring assembly.

### EXAMPLE 18

The catheter according to Example 15, wherein distal ends of the splines are connected to a cylindrical or funnel shaped element configured at a distal end of the coil-spring assembly.

### EXAMPLE 19

The catheter according to Example 15, wherein proximal ends of the splines are coupled to a proximal coupler to which a proximal end of the coil-spring assembly is coupled and a distal end of the shaft is also coupled.

### EXAMPLE 20

The catheter according to Example 15, wherein a plurality of electrodes are disposed on the splines and arranged such that in the expanded state of the expandable assembly a circumferential ablation lesion can be produced.

Those skilled in the art to which the present disclosure pertains, can appreciate that while the present disclosure has been described in terms of preferred examples, the concept upon which this disclosure is based may readily be utilized as a basis for the designing of other structures, systems and processes for carrying out the several purposes of the present disclosure.

Also, it is to be understood that the phraseology and terminology employed herein are for the purpose of description and should not be regarded as limiting. It should be noted that the words “comprising”, “including” and “having” as used throughout the appended claims are to be interpreted to mean “including but not limited to”. The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases, and disjunctively present in other cases. The term “each” may not be exclusively understood as referring to each and every, and when technically relevant may also refer to “at least some”.

All patents and patent applications mentioned in this specification are herein incorporated in their entirety by reference into the specification, to the same extent as if each individual patent or patent application was specifically and individually indicated to be incorporated herein by reference. In addition, citation or identification of any reference in this application shall not be construed as an admission that such reference is available as prior art to the present disclosure.

It is important, therefore, that the scope of the present disclosure is not construed as being limited by the illustrative examples set forth herein. Other variations are possible within the scope of the present disclosure as defined in the appended claims. Other combinations and sub-combinations of features, functions, elements and/or properties may be claimed through amendment of the present claims or presentation of new claims in this or a related application. Such amended or new claims, whether they are directed to different combinations or directed to the same combinations, whether different, broader, narrower or equal in scope to the original claims, are also regarded as included within the subject matter of the present description.

1. A catheter comprising:
a shaft configured for insertion into a body of a subject;
an expandable assembly mounted at a distal end of the shaft and comprising a proximal end, a distal end, and a longitudinal axis extending between the proximal and distal ends;
a coil-spring assembly extending between the proximal end and the distal end of the expandable assembly, along the longitudinal axis; the coil spring assembly comprising at least two nested coil springs which define an inner passageway for an elongate element to be passed therethrough; the coil-spring assembly being configured to deflect along with the expandable assembly while the inner passageway is maintained open.

2. The catheter according to claim 1, wherein the coil-spring assembly constitutes the only direct coupling between the proximal and distal ends of the expandable assembly which extends centrally along the longitudinal axis.

3. The catheter according to any one of the preceding claims, wherein the passageway defined by the coil-spring assembly is sized to enable an elongate element in the form of a guidewire to be passed therethrough.

4. The catheter according to claim 1, wherein the coil-spring assembly comprises a lefthanded coiled spring and a righthanded coiled spring.

5. The catheter according to claim 4, wherein a pitch of each of the coil springs is sized so that lateral gaps of the coil-spring assembly are small enough to prevent even a thin elongate element from protruding therethrough.

6. The catheter according to claim 1, wherein deflection of the coil-spring assembly and the expandable assembly is with respect to the longitudinal axis.

7. The catheter according to claim 1, wherein the coil-spring assembly is at least partially compressible and extensible with respect to the longitudinal axis.

8. The catheter according to claim 1, wherein the inner passageway has a circular cross section profile.

9. The catheter according to claim 1, wherein the coil-spring assembly is integrally formed with the expandable assembly.

10. The catheter according to claim 1, wherein the coil-spring assembly is coupled to the proximal and distal ends of the expandable assembly via cylindrical or funnel-shaped members.

11. The catheter according to claim 1, wherein the coil-spring assembly is produced by laser-cutting a nitinol tube.

12. The catheter according to claim 1, wherein the expandable assembly comprises a plurality of electrodes for delivery of ablation energy being mounted thereon.

13. The catheter according to claim 12, wherein the electrodes are configured for pulsed field ablation or RF ablation.

14. The catheter according to claim 1, further comprising a plurality of position sensors disposed on one or both of the shaft and the expandable assembly for tracking a position and an orientation of at least a portion of the catheter.

15. The catheter according to claim 1, wherein the expandable assembly comprises a basket including a plurality of splines, each spline connected to the proximal end and to the distal end; the basket being manipulable between an expanded state in which the splines bow radially outwardly with respect to the longitudinal axis, and a collapsed state in which the splines are aligned parallel to the longitudinal axis.

16. The catheter according to claim 15, wherein in the collapsed state the catheter is sized to be delivered through a delivery sheath; and wherein upon extending the expandable assembly distally from the delivery sheath, the coil-spring assembly extends distally.

17. The catheter according to claim 15, wherein in the collapsed state the plurality of splines extend parallel to the longitudinal axis and thereby parallel to the coil-spring assembly.

18. The catheter according to claim 15, wherein distal ends of the splines are connected to a cylindrical or funnel shaped element configured at a distal end of the coil-spring assembly.

19. The catheter according to claim 15, wherein proximal ends of the splines are coupled to a proximal coupler to which a proximal end of the coil-spring assembly is coupled and a distal end of the shaft is also coupled.

20. The catheter according to claim 15, wherein a plurality of electrodes are disposed on the splines and arranged such that in the expanded state of the expandable assembly a circumferential ablation lesion can be produced.
